(19) United States
(12) Patent Application Publication
(10) Pub. No.: US 2019/0209110 A1
(43) Pub. Date: Jul. 11, 2019

(54) Cassette Cover With Zip-Lock

(71) Applicant: Precision Dynamics Corporation, Valencia, CA (US)

(72) Inventors: Jennifer Thompson, Saugus, CA (US); Jessie Rios, Saugus, CA (US)

(21) Appl. No.: 16/240,363

(22) Filed: Jan. 4, 2019

Related U.S. Application Data
(60) Provisional application No. 62/614,556, filed on Jan. 8, 2018.

(51) Int. Cl. A61B 6/00; G03B 42/04; B65D 33/25

(52) U.S. Cl. CPC A61B 6/4423; G03B 42/04; B65D 33/2508; A61B 6/4411; A61B 6/4283

(57) ABSTRACT

A cover for an x-ray cassette includes a zip-lock. This x-ray cassette cover may be implemented in a method of protecting and/or using an x-ray cassette and may be part of a sanitation system. The zip-lock provides for easy sealing and removal of the x-ray cassette from the cover.

### CROSS-REFERENCE TO RELATED APPLICATION

This application claims the benefit of U.S. Provisional Patent Application No. 62/614,556 entitled “Zip Top Cassette Cover” filed on Jan. 8, 2018, the entire contents of which are incorporated herein by reference.

### TECHNICAL FIELD

The present disclosure is directed to a protective barrier or cover for x-ray cassettes.

### BACKGROUND

X-ray cassettes are rigid holders for holding x-ray film systems and intensifying screens and for protecting them prior to and during use. Most x-ray cassettes are rectangular in shape, although they might be, in some forms, curved depending on the particular use case. Still further, many newer x-ray cassettes may be designed to incorporate digital imaging elements rather than traditional film elements, may incorporate wireless functionality, and may be portable for easy handling.

In many use cases, the x-ray cassettes are used for bedside x-rays when the patient may lack mobility. Bedside x-rays can require that the x-ray cassette be placed underneath the patient prior to imaging. With this placement, the x-ray cassette could potentially be exposed to contamination and/or bodily fluids which could damage and/or contaminate the x-ray cassettes. Still further, if the x-ray cassette becomes contaminated, then it could potentially serve to expose a subsequent patient using that x-ray cartridge to contamination from an earlier patient.

### SUMMARY

To avoid contamination concerns, protective covers have been designed to go over x-ray cassettes. For example, U.S. Pat. Nos. 4,961,502 and 3,829,699 describe such protective devices. However, such protective devices are rigid, costly, and non-disposable, so they have to be properly cleaned between uses. U.S. Pat. No. 5,123,535 describes a plastic, flexible enclosure for x-ray cassette holders. However, the cassette covers disclosed in that patent are sterile, and therefore expensive, and further still utilize an adhesive closure making them difficult to manage by a gloved technician. Highly sterile covers are not necessary for bedside patient x-rays because the environment around the patient typically does not require that level of cleanliness and it is only cross-contamination that may sought to be avoided.

Disclosed herein is a flexible, radiolucent protective barrier for mobile x-ray cassettes which is efficient to use due to the incorporation of a zip-lock closure mechanism that allows easy opening and closure of the opening of the cover. Moreover, the material of the cover can be a material which can be relatively flexible and slippery (which makes loading and unloading the cassette into the cover easy to perform), which can aid in limiting cross-contamination, and which does not meaningfully interfere with the production of a radiograph image when the x-ray cassette is in use.

According to one aspect, a method of covering an x-ray cassette is disclosed. An x-ray cassette is placed into an interior volume of a cover in which the cover includes a bag made of radiolucent material defining an interior volume sized and shaped to receive the x-ray cassette and a selectively sealable opening. A zip-lock is present at the selectively sealable opening with an interlocking groove and ridge that form a tight seal when pressed together. The cover with the x-ray cassette received therein is then sealed by pressing the interlocking groove and ridge of the zip-lock together.

In some forms, the method may further include the steps of placing the cover and x-ray cassette underneath a patient after the x-ray cassette is sealed in the cover and removing the cover and x-ray cassette from underneath the patient after imaging. After the cover and x-ray cassette are removed from underneath the patient, the x-ray cassette may be removed from the cover and the cover may be disposed of.

According to another aspect, a sanitation system for an x-ray cassette is provided. The sanitation system includes an x-ray cassette and a cover. The cover is a bag made of radiolucent material defining an interior volume sized and shaped to receive the x-ray cassette and includes a selectively sealable opening. A zip-lock is present at the selectively sealable opening with an interlocking groove and ridge that form a tight seal when pressed together.

According to still another aspect, the x-ray cassette cover described above is provided apart from the x-ray cassette.

In some forms of the method, system, or cover, the cover may be disposable after a single use (i.e., not reused in subsequent imaging operations, especially across multiple patients).

In some forms of the method, system, or cover, the radiolucent material may be high-density polyethylene and/or the radiolucent material may be formed to a thickness that is an effective viral barrier.

In some forms of the method, system, or cover, the bag may be quadrilateral in shape with one edge of the quadrilateral being the selectively sealable opening with the zip-lock. Still further, such quadrilateral may be rectangular in shape. For example, the bag may be sized and shaped to receive an x-ray cassette having length and width dimensions selected from the group consisting of 14 inches by 17 inches, 14 inches by 14 inches, 6 inches by 12 inches, 14 inches by 33 inches, and 14 inches by 49 inches.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE DRAWINGS

FIG. 1 shows an example x-ray cassette cover and an x-ray cassette partially inserted into the cover.

FIG. 2 shows a covered x-ray cassette being placed underneath a patient.

FIG. 3 shows a sanitation system for an x-ray cassette.

### DETAILED DESCRIPTION

An x-ray cassette is a light-tight container in which x-ray films are placed for exposure to ionizing radiation and can be backed by lead to eliminate the effects of back scatter radiation. In medicine, “radiolucent” characterizes something that allows passage of x-rays or other radiation.

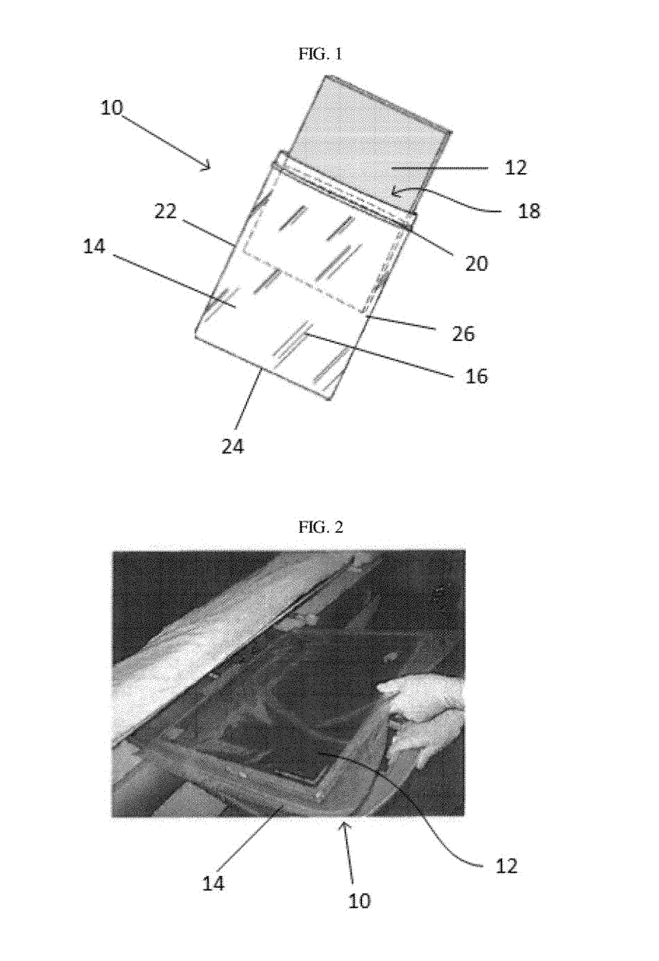

FIG. 1 is an example x-ray cassette cover according to an embodiment of the invention. The x-ray cassette cover 10 comprises a bag 14 with an interior volume 16, which is sized and shaped to receive an x-ray cassette 12. The bag 14 includes a selectively sealable opening 18, the selectively sealable opening 18 comprises an interlocking groove and ridge providing a zip-lock 20 that forms a tight seal when pressed together. It is contemplated that the selectively sealable opening 18 could be configured in other forms to provide a tight seal, such forms including a toothed zipper, a slidable bag lock that presses an interlocking groove and ridge together when slid across the length of the selectively sealable opening 18, among others.

The bag 14 shown in FIG. 1 is a generally-rectangular quadrilateral shape having three sides 22, 24, and 26 that are permanently sealed. As such, the bag 14 has two sets of parallel edges. Most typically x-ray cassettes are rectangular; however, other bag 14 shapes are contemplated that correspond to a variety of possible x-ray cassette shapes including rectangles curved to from a semi-tubular shape or other geometric profiles, wherein the bag 14 has permanently sealed edges except for a selectively sealable opening 18 sized to allow entry of a corresponding x-ray cassette geometry. The dimensions of the bag 14 and its opening 18 are sufficient to fit at least an x-ray cassette 12. Still further, it will be appreciated that the profile of the bag does not exactly match the shape of the x-ray cassette; for example, there may be slightly rounded corners on one of the bag and/or cartridge and further there is likely to be at least some clearance between the bag and cartridge.

With respect to the bag 14 having a quadrilateral shape as shown in FIG. 1, the bag 14 can be, for example, but not limited to, a variety of dimensions including length and width dimensions of 14 inches by 17 inches, 14 inches by 14 inches, 6 inches by 12 inches, 14 inches by 33 inches, and 14 inches by 49 inches among others. The foregoing bag 14 dimensions correspond to the variety of x-ray cassette sizes commercially available for use in procuring x-ray images.

Referring further to FIG. 1, the bag 14 is comprised of a radiolucent material. In a preferred embodiment, the bag 14 comprises high-density polyethylene (“HDPE”). HDPE has a very low coefficient of friction to assist in sliding the x-ray cassette 12 into the cover 10, in sliding the x-ray cassette cover 10 underneath a patient, and further is flexible enough to accommodate manufacture a zip-lock closure. HDPE is also sturdy enough to hold the weight of an x-ray cassette. HDPE is also an effective viral barrier and the thickness of bag 14 can be optimized to ensure that viral material cannot penetrate through the exterior of the bag 14 into the interior volume 16 of the bag 14. HDPE is also sufficiently radiolucent to not produce artifacts in x-ray images taken with typical x-ray settings used to image typical patients in a healthcare setting.

Starting from an uncovered x-ray cassette 12, a method of covering an x-ray cassette 12 using a cover 10 is now described as well as the subsequent use of the covered cassette.

To place the x-ray cassette 12 in the cover 10, the opening 18 of the cover 10 is first opened, if necessary. As noted above, the zip-lock 20 serves to open or seal the opening 18 and so this zip-lock 20 must be opened (if not already opened) by separating the interlocking groove and ridges from one another. With the interior volume 16 of the bag 14 being now accessible via the opening 18, the x-ray cassette 12 is placed into the corresponding interior volume 16 of the bag 14 of the x-ray cassette cover 10. Once the x-ray cassette 12 is fully received within the cover 10, the cover 10 is then sealed by closing the zip-lock 20 by pressing the interlocking groove and ridge together.

FIG. 2 shows a covered x-ray cassette being placed underneath a patient. “Slippery” bag material which is manufactured with a low coefficient of friction makes it easier for the covered x-ray cassette to be placed underneath a patient. The bag 14 can be comprised of a material with a coefficient of friction, for example, of less than 0.4 (HDPE is typically around 0.28).

With the x-ray cassette 12 received in a cover 10 beneath the patient, an x-ray technician or other healthcare provider operates an x-ray machine so that the x-ray image is captured on or using the x-ray cassette 12. The x-ray cassette 12 can then be removed from underneath the patient and the x-ray cassette 12 removed from the cover 10, resulting in the x-ray cassette 12 remaining free from patient contaminants during use, including contaminates such as fluids or other particulates that might otherwise come into contact with the x-ray cassette 12 without the use of the x-ray cassette cover 10. The cover 10 can be disposed (most likely after a single use), depending on the required conditions dictated by the healthcare provider, the type of media in the x-ray cassette, and whether subsequent images are taken of the same or a different patient.

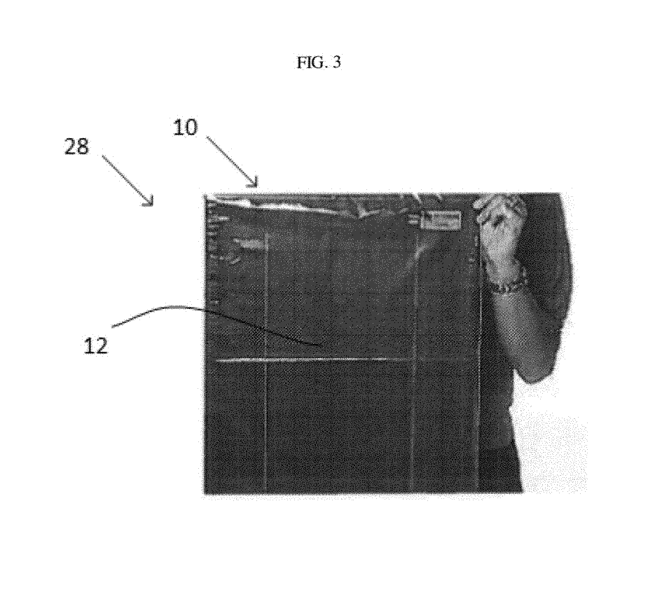

FIG. 3 shows an x-ray cassette sanitation system 28. The sanitation system 28 includes an x-ray cassette 12 and an x-ray cassette cover 10 according to one embodiment of the invention, wherein the x-ray cassette cover 10 is disposable after a single use. This disposable cover 10 allows technicians to move from one patient to another quickly, replacing the cover 10 efficiently between patients.

The preceding disclosure is not meant to be exhaustive or to limit the scope of the invention to the form factors or use cases disclosed, but rather serve to illustrate and describe preferred embodiments. Other embodiments within the principles and practices of the preceding disclosure may be apparent to those skilled in the art. All suitable modifications and equivalents falling within the scope of the invention may be implemented.

1. A method of covering an x-ray cassette, the method comprising:
placing an x-ray cassette into an interior volume of a cover, in which the cover comprises a bag made of a radiolucent material defining an interior volume sized and shaped to receive the x-ray cassette, in which the bag includes a selectively sealable opening, the bag further comprising a zip-lock at the selectively sealable opening with an interlocking groove and ridge that form a tight seal when pressed together;
sealing the cover with the x-ray cassette received therein by pressing the interlocking groove and ridge of the zip-lock together.

2. The method of claim 1, further comprising the step of placing the cover and x-ray cassette underneath a patient after the x-ray cassette is sealed in the cover and removing the cover and x-ray cassette from underneath the patient after imaging.

3. The method of claim 2, further comprising the step of removing the x-ray cassette from the cover after the cover has been removed from underneath the patient and disposing of the cover.

4. The method of claim 3, wherein the disposing of the cover occurs after a single instance of placing the cover and x-ray cassette underneath a patient after the x-ray cassette is sealed in the cover and removing the cover and x-ray cassette from underneath the patient after imaging.

5. The method of claim 1, wherein the radiolucent material is high-density polyethylene.

6. The method of claim 1, wherein the radiolucent material is formed to a thickness that is an effective viral barrier.

7. The method of claim 1, wherein the bag is sized and shaped to receive an x-ray cassette having length and width dimensions selected from the group consisting of 14 inches by 17 inches, 14 inches by 14 inches, 6 inches by 12 inches, 14 inches by 33 inches, and 14 inches by 49 inches.

8. A sanitation system for an x-ray cassette, the sanitation system comprising:
an x-ray cassette; and
a cover comprising a bag made of a radiolucent material, sized and shaped to receive the x-ray cassette in which the bag defines an interior volume having a selectively sealable opening and further comprising a zip-lock at the selectively sealable opening with an interlocking groove and ridge that form a tight seal when pressed together.

9. The sanitation system of claim 8, wherein the cover is disposable after a single use.

10. The sanitation system of claim 8, wherein the radiolucent material is high-density polyethylene.

11. The sanitation system of claim 8, wherein the radiolucent material is formed to a thickness that is an effective viral barrier.

12. The sanitation system of claim 8, wherein the bag is sized and shaped to receive an x-ray cassette having length and width dimensions selected from the group consisting of 14 inches by 17 inches, 14 inches by 14 inches, 6 inches by 12 inches, 14 inches by 33 inches, and 14 inches by 49 inches.

13. An x-ray cassette cover comprising:
a bag made of a radiolucent material, sized and shaped to receive an x-ray cassette in which the bag defines an interior volume having a selectively sealable opening;
a zip-lock at the selectively sealable opening with an interlocking groove and ridge that form a tight seal when pressed together.

14. The x-ray cassette cover of claim 13, wherein the radiolucent material is high-density polyethylene.

15. The x-ray cassette cover of claim 13, wherein the radiolucent material is formed to a thickness that is an effective viral barrier.

16. The x-ray cassette cover of claim 13, wherein the bag is quadrilateral in shape with one edge of the quadrilateral being the selectively sealable opening with the zip-lock.

17. The x-ray cassette cover of claim 13, wherein the bag is sized and shaped to receive an x-ray cassette having length and width dimensions selected from the group consisting of 14 inches by 17 inches, 14 inches by 14 inches, 6 inches by 12 inches, 14 inches by 33 inches, and 14 inches by 49 inches.
